(19) United States
(12) Patent Application Publication
(10) Pub. No.: US 2014/0200446 A1
(43) Pub. Date: Jul. 17, 2014

(54) Method and apparatus for the infusion of a catheter into an artery/vein and for suspected tissue removal

(71) Applicant: Thomas Haider, Rancho Santa Fe, CA (US)

(72) Inventor: Thomas Haider, Rancho Santa Fe, CA (US)

(21) Appl. No.: 13/694,904

(22) Filed: Jan. 16, 2013

(51) Int. Cl. A61B 5/00; A61B 10/04; A61B 10/02

(52) U.S. Cl. CPC A61B 5/0084; A61B 5/0066; A61B 10/0233; A61B 10/04; A61B 5/6852
USPC 600/427

(57) ABSTRACT

An instrument adapted for the infusion of a catheter into a vessel or performing a biopsy procedure is in the form of a needle with an OCT probe affixed adjacent its distal end. An optics cable extends from the probe to an optoelectronics module which supplies light to, receives reflected light from the probe and deciphers the reflected light to form an image of the tissue 2-3 mm in from to the probe. A catheter may be placed over the instrument for insertion into a vessel or vein or the instrument may be provided with a tissue receiving channel for biopsy use.

### RELATED APPLICATION

This application is based on and claims priority of U.S. Provisional Application No. 61/632,352, filed on Jan. 23, 2012, the contents of which are incorporated herein by reference.

### FIELD OF THE INVENTION

The present invention relates to an improved method and apparatus for infusing a catheter into a patient's artery or vein to enable a care giver to withdraw from or supply liquid nutrients, medicine and the like directly into the patient's blood stream. Additionally the invention is directed to an instrument and method for obtaining a biopsy.

### BACKGROUND OF THE INVENTION

### Catheter Infusion

Needles, with a flexible catheter slidably mounted over their distal ends, are typically used to insert or infuse a catheter into a patient's vein or artery, with the needle being withdrawn once the catheter is in place. This procedure is fraught with problems. Arteries and veins, (“vessels”), are not typically immobile but tend to move as the needle is advanced toward them. Without the nurse or doctor (installer) knowing the precise location of the vessel, in real time, the procedure often resolves itself into a trial and error situation with success being problematic. In addition, even when the installer has successfully engaged the targeted vessel with the needle, a successful outcome is not assured. While the needle may have entered the vessel, so that blood can be seen at the proximal end of the needle, the catheter may still be outside of the vessel; also, the needle and catheter may have passed entirely through the vessel.

Guide wires, inserted through the needle, have been used to guide a catheter into the interior of a vein or vessel. However, this technique is of little help to the installer in finding an elusive vessel. In addition, ultrasound equipment has been used to aid the installer in guiding a needle to an illusive vessel. None of these techniques take the place of a human eye's ability to accurately steer a needle/catheter to and into a targeted vessel.

There is a need for the installer to see, in real time, the position of the tip of the needle relative to the targeted vessel. In my research I have found the optical coherence tomography (“OCT), a cutting edge technology, is capable of providing an image in front of a probe through several millimeters of tissue. The use of this technology in conjunction with a needle/catheter will enable an installer to visualize the tissue, including a targeted vessel positioned 2 to 3 mm in front of the needle tip, thereby largely eliminating the hit or miss procedures of the prior art.

### Tissue Removal from a Suspected Abnormal Growth

Various methods have and are being used to assist a surgeon in finding and subsequently positioning a conventional biopsy needle in an appropriate place to enable a sample of the suspected abnormal growth to be removed for analysis. Also, such conventional needles and procedures leave something to be desired in determining the best places to take a sample or samples of an oddly-shaped growth. Similar to the conventional catheter infusion technique discussed above there is a need for the surgeon to be able to see the size and shape of the abnormal growth from the perspective of the front of the biopsy needle. To this end I have determined that the use of an OCT probe positioned at the front of a biopsy needle would be a great benefit to s surgeon.

### SUMMARY OF THE INVENTION

### Catheter Infusion

A method, in accordance with the present invention, includes the steps of first providing a needle with an OCT probe located at its distal end or tip with a fiber optic cable extending from the probe along the interior of the needle and exiting from near the proximal end of the needle. The exiting end of the cable is connected to an optical end electronics module capable of supplying light to, receiving reflected light from the probe, and deciphering the information contained in the reflected light. The output of the module is fed to a computer and monitor for providing the installer with an image of the tissue immediately in front of the needle.

Second, a catheter is inserted over the needle so that the distal end of the catheter is positioned near the tip of the needle. Third, the needle and catheter, so assembled, are inserted into a patient adjacent the targeted artery or vein. Fourth, while viewing the displayed image in front of the needle, the needle and catheter is guided into the targeted vessel. Fifth, the needle is withdrawn leaving the catheter in place.

### Tissue Removal

An improved biopsy needle, in accordance with this invention, comprises a double lumen or channel needle with one channel being arranged to accommodate the removal of a sample of the targeted growth. An OCT probe is positioned adjacent the distal end or tip of the other channel with an optical fiber cable extending through and exiting adjacent the proximal end of the needle. An optical and electronics module is connected to the exiting end of the cable for supplying light to, receiving reflected light from the probe and deciphering the reflected light to provide a real time image of the tissue positioned 2 to 3 mm in front of the needle.

### BRIEF DESCRIPTION OF THE DRAWINGS

FIG. 1 is a cross-sectional view of a catheter insertion needle, housing an OCT probe at its distal end, the probe being connected to an optoelectronics and optical components module and computer/monitor with a catheter positioned over the outside of the needle;

FIG. 2 is a simplified diagrammatic view of the tip of the needle/catheter of FIG. 1 being inserted into the targeted vessel;

FIG. 3 is a view, similar to FIG. 2, showing the catheter in place inside of the vessel and the needle being withdrawn;

FIG. 4 is a view of the catheter remaining in place after the needle has been withdrawn;

FIG. 5 is a cross sectional view of my improved biopsy needle;

FIG. 6 is an enlarged cross-sectional view of the distal section of the needle of FIG. 5 with a tissue removal instrument, shown for example as a corkscrew, in place in the upper channel;

FIG. 7 is an enlarged end view of the needle of FIG. 5; and

FIGS. 8a and 8b are simple diagrammatic views with FIG. 8a showing the biopsy needle having entered the patient's body and approaching a suspected abnormal growth with FIG. 8b showing the tissue removal instrument entering the growth.

### DESCRIPTION OF THE PREFERRED EMBODIMENT

### Catheter Infusion

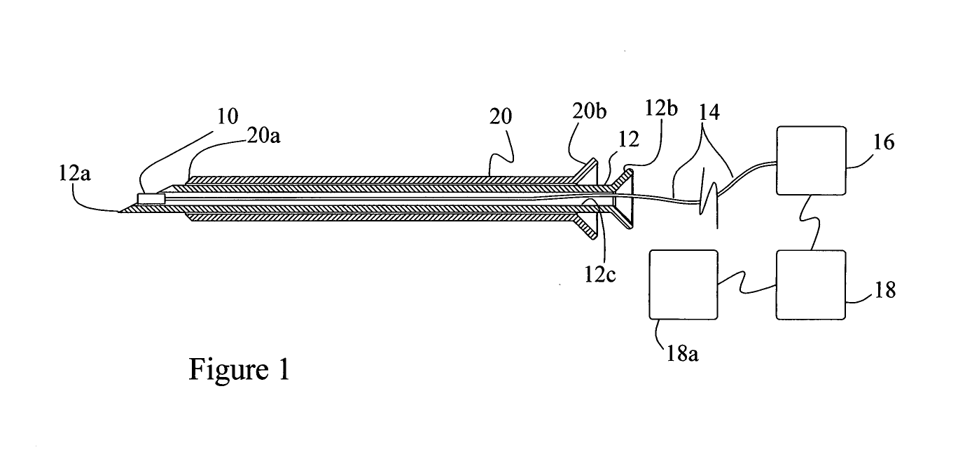

Referring now to FIG. 1 an OCT probe 10 is positioned within the tip or distal end 12a of a conventional needle 12. A fiber optics cable 14, extending through the channel 12c, connects the probe to an optoelectronics and optical components module 16 which provides a digital output to a computer (e.g. a conventional laptop 18 and associated monitor 18a). A catheter 20 having a distal end (20a) and a proximal end (20b) is slipped over the outside of the needle in a conventional manner. It should be noted that the probe 10 and cable 14 can be embedded in the needle leaving an open channel running the length of the needle.

The OCT probe may include a gradient index lens, (GRIN) or other suitable optics to direct light onto and receive reflected light from the tissue immediately in front of the needle. The module 16 supplies light to and receives the reflected light from the OCT probe. Such OCT systems for imaging in nontransparent tissue are described in several treatises. See for example, the book “Optical Coherence Tomography”, published by Elsevier and authored by Dr. Mark E Brezinski, and in particular chapter 6 and the light delivery devices in subchapter 6.7. Also see the Biomedical Optical Imaging book edited by James G. Fujimoto and Daniel L. Farkas (Oxford University Press) and in particular pages 170 and 171 for schematics of OCT probes (referred to as a catheter on page 172) and related optics and electronics for powering the probe and deciphering the information obtained there from to provide an image several millimeters deep into the tissue in front of the probe.

An article in Vol. 47, No. 8 Suppl.C, Apr. 18, 2006, of the Journal of the American College of Cardiology entitled “Plaque Characterization With Optical Coherence Tomography” by Debra Stamper et al describes and depicts in schematic form an OCT imaging engine including an OCT probe, (referred to as a catheter) of only 0.017 inches in diameter made by Lightlab of Westford, Mass.

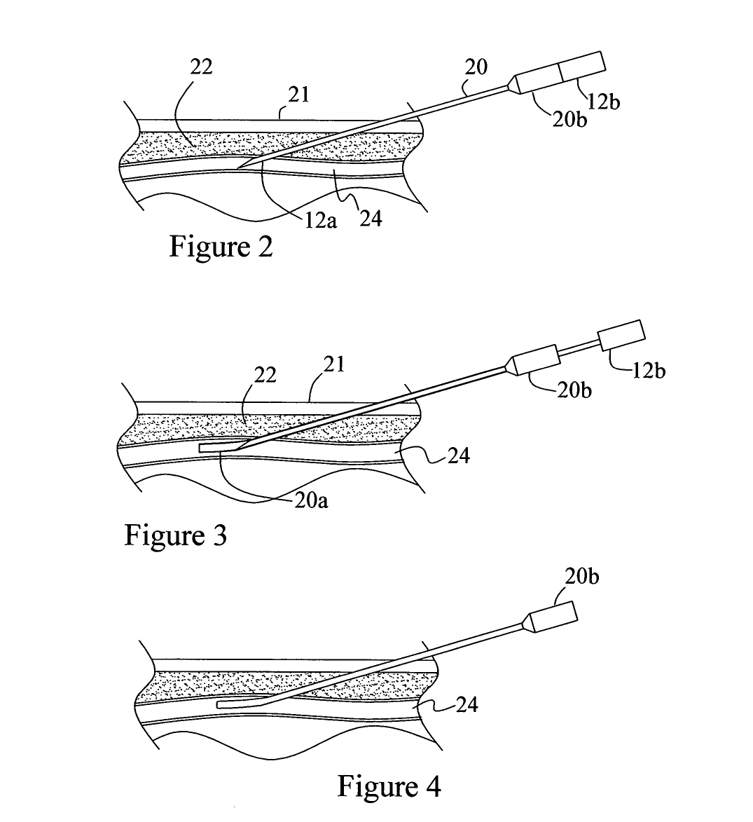

In use the care giver, while viewing an image of the tissue 2 or 3 mm in front of the tip 12a of the needle via the monitor, inserts the needle and catheter into a patient's tissue adjacent the targeted vein or artery, as is illustrated in FIG. 2. Once the catheter is placed in the artery or vein the desired distance the needle is withdrawn, as illustrated in FIG. 3, leaving the catheter in place as is illustrated in FIG. 4

### Tissue Removal

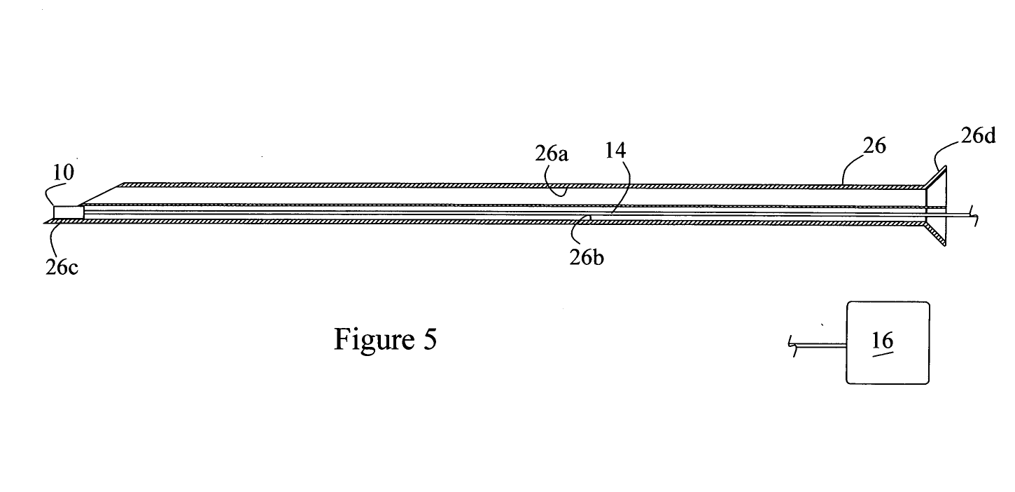

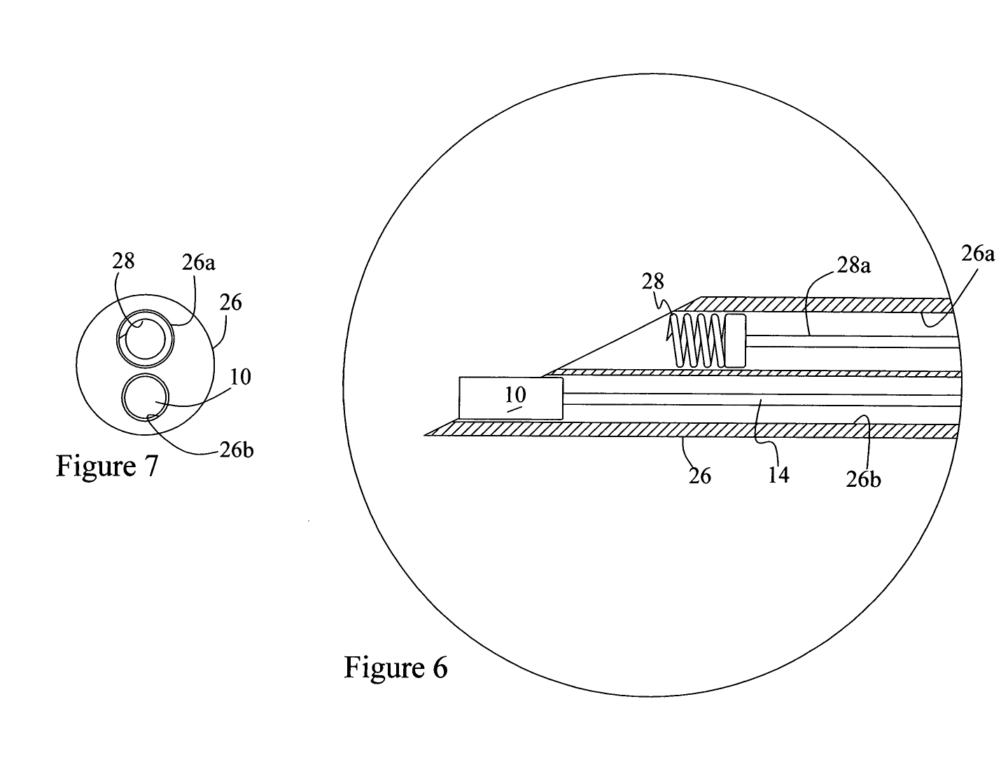

Referring now to FIGS. 5-7, an improved biopsy needle 26 is formed with two channels or lumens 26a and 26b extending the length thereof. An OCT probe 10 is positioned in the lower channel 26b, adjacent the distal end 26c, as shown. An optical cable 14 connected to the probe, exits the proximal end 26d of the channel for connection to the optoelectronics and optical module 16 shown in FIG. 1. The upper channel 26a is open for receiving a suitable tissue removal instrument, shown as simple corkscrew 28 at the end of a manually manipulative rod or shaft 28a in FIG. 6.

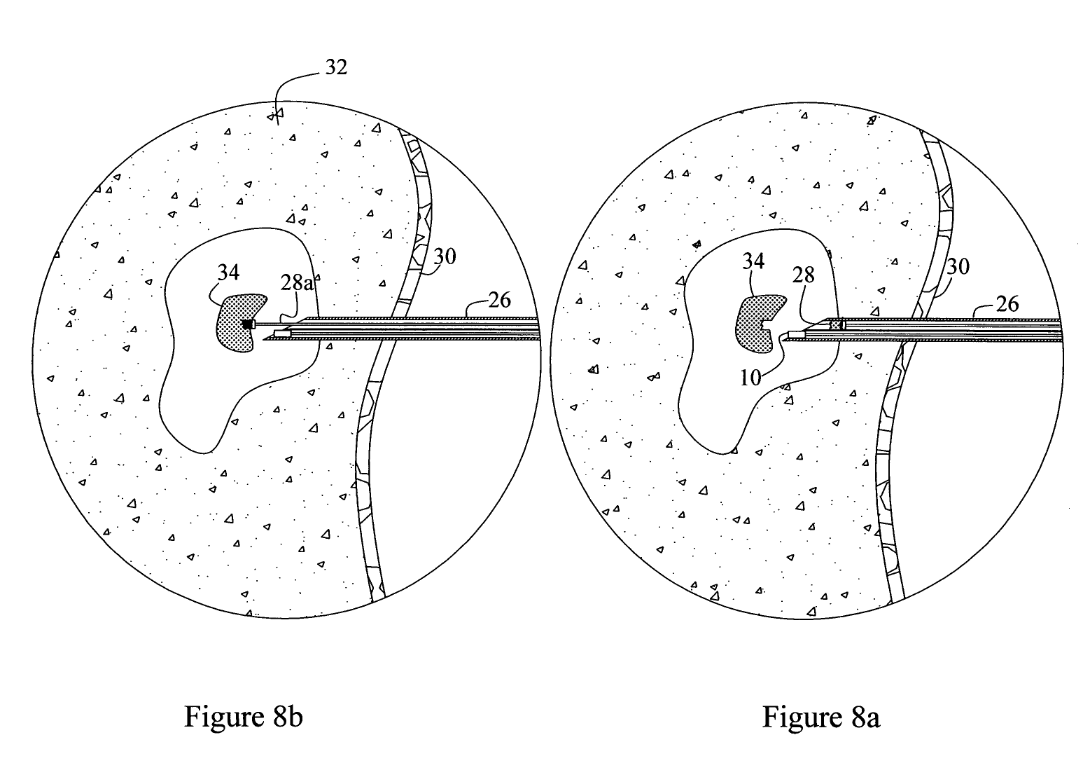

Referring now to FIGS. 8a and 8b, in use the biopsy needle of FIGS. 5-7 is inserted through a patient's skin 30 and subsurface tissue 32 toward the targeted growth 34 by the surgeon while viewing the tissue in front of the needle, via a monitor 18a. The size and configuration of the growth 34 may be ascertained through the monitor. Once a specific site has been selected the surgeon can place a tissue collecting instrument in the upper channel 26a, if not already in place, and insert it into the growth. The shaft may then be withdrawn removing the sample for analysis.

It should noted that the OCT probe and optics cable may be formed as an integral part of the needles eliminating the need for a separate channel.

What is claimed is:

1. An instrument adapted for the infusion of a catheter into a vessel or performing a biopsy procedure comprising a needle having a proximal and distal end, an OCT probe affixed adjacent its distal end with an optics cable extending from the probe and exiting the needle adjacent the proximal end, the cable being connected to an optoelectronics and optical module capable of supplying light to, receiving reflected light from the probe and deciphering the reflected light to provide information representative of an image of the tissue several mm in front of the distal end of the needle.

2. The instrument of claim 1 wherein the needle has a central opening running the length thereof with the probe and section of the optics cable positioned within the channel.

3. The instrument of claim 2 wherein the needle includes two channels running the length thereof with the probe and optics cable positioned in one of the channels, the other channel being arranged to receive a tissue removal instrument.

4. A method for infusing a catheter into a blood vessel comprising:
a) providing a needle with an OCT probe located at its distal end and a fiber optics cable extending from the probe along the interior of the needle and the exiting end of the cable being connected to an optoelectronics/optical components module capable of supplying light to and receiving reflected light from the probe to provide information representing an image of the tissue in front of the probe,
b) placing a catheter over the needle so that the distal end of the catheter is positioned adjacent the distal end of the needle,
c) inserting the needle and catheter into a patient adjacent a targeted vessel,
d) viewing the image in front of the needle while guiding the needle to the targeted vessel,
e) inserting the needle and catheter into the targeted vessel, and
f) withdrawing the needle while leaving the catheter in place;

5. An improved biopsy needle having proximal and distal ends, the needle being formed with an OCT probe disposed adjacent its distal end with an optics cable connected at one end to the probe , the other end of the optics cable exiting the cable and adapted to be connected to an optoelectronics and optical module capable of supplying light to, receiving light from the probe and deciphering the received light into a representation of an image of the tissue several mm in front of the proximal end of the needle, and a tissue receiving channel extending the length of the needle, the tissue receiving channel being adapted to accommodate a tissue collecting instrument and tissue of suspected abnormal growth removed thereby.

6. The biopsy needle of claim 5 wherein the needle is formed with a separate OCT channel in which the OCT probe is housed.

7. The biopsy needle wherein the tissue receiving channel is disposed below the OCT channel.

8. A method of obtaining a sample of tissue from a suspected abnormal growth comprising:
a) providing a needle as defined in claim 5,
b) inserting the needle into a patient, while guiding the needle toward the growth with aid of the image,
c) inserting the needle into a selected site on the growth, and
d) removing a sample of the growth with the aid of a tissue removal instrument positioned in said one channel.
